(19) United States
(12) Patent Application Publication
(10) Pub. No.: US 2006/0211917 A1
(43) Pub. Date: Sep. 21, 2006

(54) Data generating device and endoscope system

(75) Inventors: Yosuke Ikemoto, Yamaguchi (JP); Taro Nakasendo, Saitama (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/362,772

(22) Filed: Feb. 28, 2006

(30) Foreign Application Priority Data
Mar. 1, 2005 (JP) P2005-056072

(51) Int. Cl. A61B 1/00; G09B 23/28; A61B 1/04

(52) U.S. Cl. 600/118; 434/262

(57) ABSTRACT

A data generating device for endoscope observation includes an annular insertion simulator and a moving amount data generator. The annular insertion simulator simulates insertion of an endoscope into a subject. The moving amount data generator generates moving amount data representing a virtual moving amount of the annular insertion simulator during a simulated insertion, based on an actual operation amount of the annular insertion simulator.

### BACKGROUND OF THE INVENTION

1. Field of the Invention

The present invention relates to a data generating device for endoscope observation and an endoscope system, especially it relates to a data generating device which generates data for selecting an image of a predetermined part of a subject from all the images of the subject previously stored, by operations similar to those of a typical endoscope, and an endoscope system where a predetermined part of a subject can be easily selected from all the images of the subject.

2. Description or the Related Art

Generally, an endoscope system where a three-dimension image of a subject can be displayed by processing image data obtained by an electronic endoscope, an X-ray CT device, MRI device, and so on is known. On the other hand, a capsule endoscope where a series of images of a subject are generated by a computer, by processing image data obtained by a capsule which is for photographing inside a body and is swallowed by a subject person, has been developed.

In such endoscope systems where a series of images of a subject can be displayed, an image of a desired part of a subject is selected and displayed by operations of a key board, a control stick, and so on. Those operations are totally different from the operations of a typical endoscope device which users are familiar with, therefore, it is sometimes difficult for users to promptly select an image of a desired part of a subject.

### SUMMARY OF THE INVENTION

Therefore, an objective or the present invention is to provide a data generating device which can generate data for selectively generating an image of a predetermined part of a subject from a series of images of the subject, by an operation similar to that of typical endoscopes, and to provide an endoscope system which can easily select and generate an image of a predetermined part of a subject.

A data generating device according to the present invention, is for endoscope observation and includes an annular insertion simulator which simulates insertion of an endoscope into a subject and a moving amount data generator. The moving amount data generator generates moving amount data representing a virtual moving amount of the annular insertion simulator during a simulated insertion, based on an actual operation amount of the annular insertion simulator.

In the data generating device, the moving amount data can represent a virtual moving amount of the annular insertion simulator from a reference position. The annular insertion simulator can be an endless loop, and a part of the annular insertion simulator can be linear.

The data generating device can include an operation amount detector that detects the actual operation amount of the annular insertion simulator. The operation amount detector may detect amount of rotation of the annular insertion simulator as the actual operation amount, and the moving amount data generator may generate the moving amount data by multiplying a predetermined coefficient by the amount of rotation.

The data generating device can include a direction data generator that generates direction data representing a virtual direction of a virtual tip of the annular insertion simulator for observing the subject. The data generating device can further include a data outputting device that outputs the moving amount data and the direction data to an external device, or a direction adjuster that adjusts the virtual direction.

The direction adjuster may include an angle knob to simulate an operation for adjusting a direction of the virtual tip. In the data generating device, the annular insertion simulator may be provided close to the direction adjuster so that the annular insertion simulator and the direction adjuster can be operated by a user at the same time.

The data generating device can include a mode selector that selects one of a first mode where the moving amount data generator generates the moving amount data based on the actual operation amount of the annular insertion simulator, and a second mode where the moving amount data generator generates the moving amount data based on a proportional operation amount of the annular insertion simulator, the proportional operation amount being proportional to the actual operation amount. The proportional operation amount can be smaller than the actual operation amount.

An endoscope system according to the present invention includes an annular insertion simulator which simulates insertion of an endoscope into a subject, a moving amount data generator, a position data generator, an image storage, and an image generator. The moving amount data generator generates moving amount data representing a virtual moving amount or the annular insertion simulator during a simulated insertion, based on an actual operation amount of the annular insertion simulator. The position data generator generates position data representing a position of a virtual tip of the annular insertion simulator for observing the subject, based on a predetermined reference position of the virtual tip and the moving amount data. The image storage stores image data of the subject, and the image generator generates a partial image of the subject by processing the image data. The image generator generates a partial image corresponding to a position of the virtual tip, the position being represented by the position data, and the partial image being supposed to observe.

The endoscope system can include an image display that displays the partial image.

### BRIEF DESCRIPTION OF THE DRAWINGS

The present invention will be better understood from the description of the preferred embodiments of the invention set forth below together with the accompanying drawings, in which:

FIG. 1 is a side view of a data generating device of the first embodiment;

FIG. 2 is a perspective view of the data generating device of the first embodiment;

FIG. 3 is a front view of the data generating device of the first embodiment;

FIG. 4 is a plan view of the data generating device of the first embodiment;

FIG. 5 is a block diagram of the electronic endoscope of the first embodiment;

FIG. 6 is a side view of a data generating device of the second embodiment;

FIG. 7 is a side view of the data generating device of the second embodiment excluding the insertion simulator;

FIG. 8 is a perspective view of the data generating device of the second embodiment excluding the insertion simulator;

FIG. 9 is a front view of the data generating device of the second embodiment excluding the insertion simulator; and

FIG. 10 is a plan view of the data generating device of the second embodiment excluding the insertion simulator.

### DESCRIPTION OF THE PREFERRED EMBODIMENTS

Hereinafter, the preferred embodiments of the present invention are described with reference to the attached drawings.

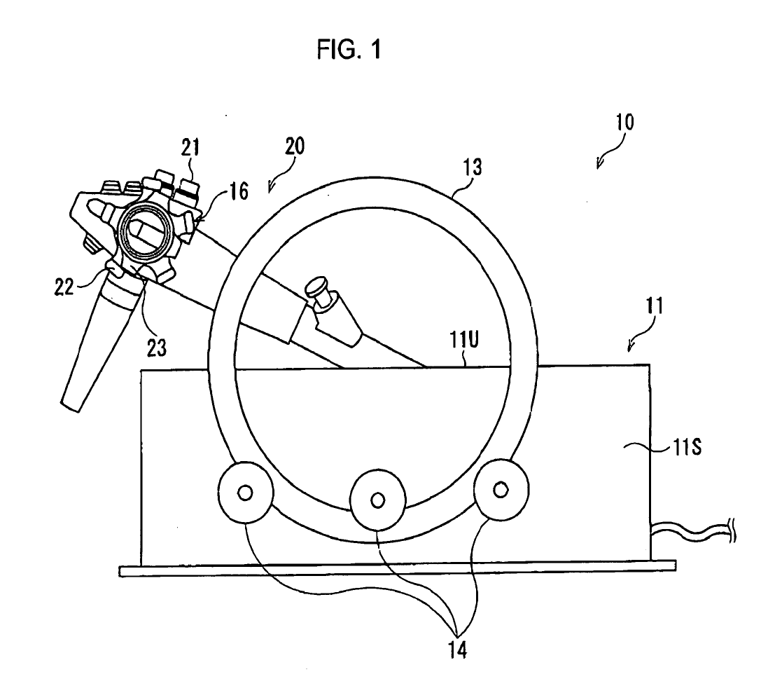

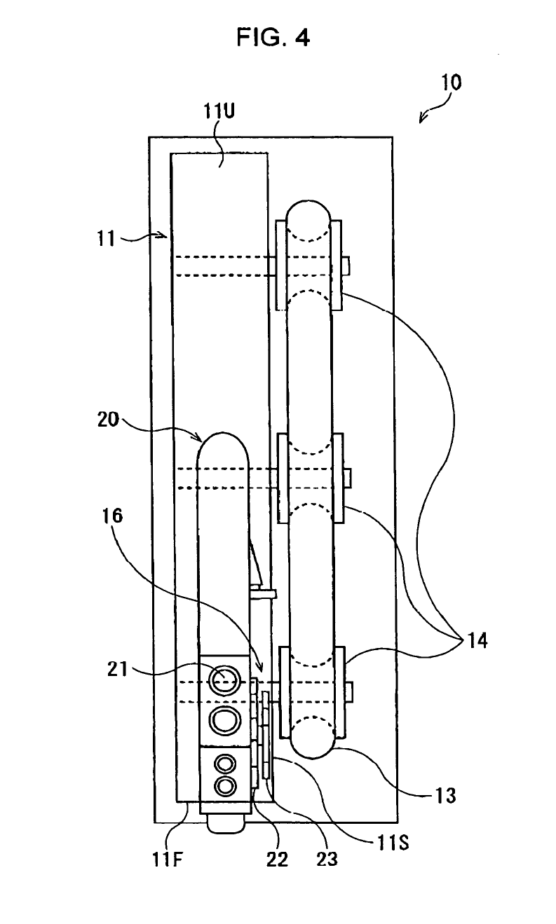

In FIGS. 1 to 4, the data generating devise 10 of the first embodiment is shown.

The data generating device 10 is used with a typical image processing device of a capsule endoscope system (not shown) here. In the image processing device, a series or video images of a subject such as a digestive organ and so on, photographed by a capsule passing through a body, is stored. The image processing device can generate the three-dimension image (hereinafter 3-D image) of a predetermined part or a subject which a user wants to observe, from the series of images of the subject previously stored. The data generating device 10 generates data for informing the image processing device of a part of the subject where the 3-D image will be generated, that is, a part of the subject which a user wants to observe.

The data generating device 10 is designed to have a shape and function similar to the shape and function of an operational part of a scope of a typical endoscope. That is, in the data generating device 10, an insertion simulator which has similar shape and function to the shape and function of an insertion tube of a real scope of a typical endoscope, is provided. Therefore, a movement of a virtual tip of the insertion simulator corresponding to the real scope, is simulated by an operation of the insertion simulator which has a similar operation to that of a real tip of the scope for inserting into a body and moving inside the body, to observe and photograph a subject with a typical endoscope. The data generating device 10 generates moving amount data representing a virtual moving amount of the virtual tip of the insertion simulator by an operation of the insertion simulator, and direction data representing a virtual direction of the virtual tip. The generated moving amount data and the direction date are transferred to an image processing device for calculating a coordinate system position and a direction of the virtual tip after the operation, and for selecting a predetermined part of the subject which the virtual tip can observe, by using the image processing device.

In the data generating device 10, an annular first endless member 13 that has a loop shape and that is rotational, as an insertion simulator and a scope simulator 20 which simulates a scope of a typical endoscope, are provided. The first endless member 13 is provided along a side surface 11S of a casing 11 of the data generating device 10. When the first endless member 13 rotates, the virtual tip of a scope moves to a predetermined part of a subject. The first endless member 13 is designed to have the same thickness and quality as an insertion tube of a scope. That is, the first endless member 13 is to simulate an operation of a real tip of an insertion tube of a scope to move to a predetermined part of a subject, by a rotation thereof. A user gradually rotates the first endless member 13 instead of operating a real scope in the data generating device 10. And then, signals corresponding to a virtual moving amount of the virtual tip of the scope which is moved by the rotation of the first endless member 13, are transferred to the image processing device.

As described above, simulating movement of the real tip of the insertion tube the scope, that is, moving the virtual tip of the scope is possible, so that a long scope used in a typical endoscope is not required, and the structure of the data generating device is simplified. Note that the first endless member 13 is supported by a pulley 14, and a frictional member 11A having a predetermined friction coefficient is provided on a pivot bearing of the pulley 14. Therefore, when the first endless member 13 rotates, a resistance for inserting an insertion tube of a real scope into body is simulated.

In the scope simulator 20 which is provided on an upper surface 110 of the casing 11, an angle knob 16 to simulate a bending operation of the virtual tip of the scope for adjusting a direction of the tip, and a release switch 21 are provided. The angle knob 16 includes a first knob 22 and a second knob 23. The first knob 22 is used for simulating an adjustment in the up and down direction of the virtual tip, and the second knob 23 is used for simulating an adjustment in the left and right direction. A user can hold the scope simulator 20 by his left hand, and can operate the angle knob 16 or the first endless member 13 with his left or right hand. The first endless member 13 is provided close to the angle knob 16 so that the first endless member 13 and the angle knob 16 can be operated by a user at the same time. The release switch 21 is used for storing a subject image displayed on the monitor (not shown) for the purpose of printing the image and so on.

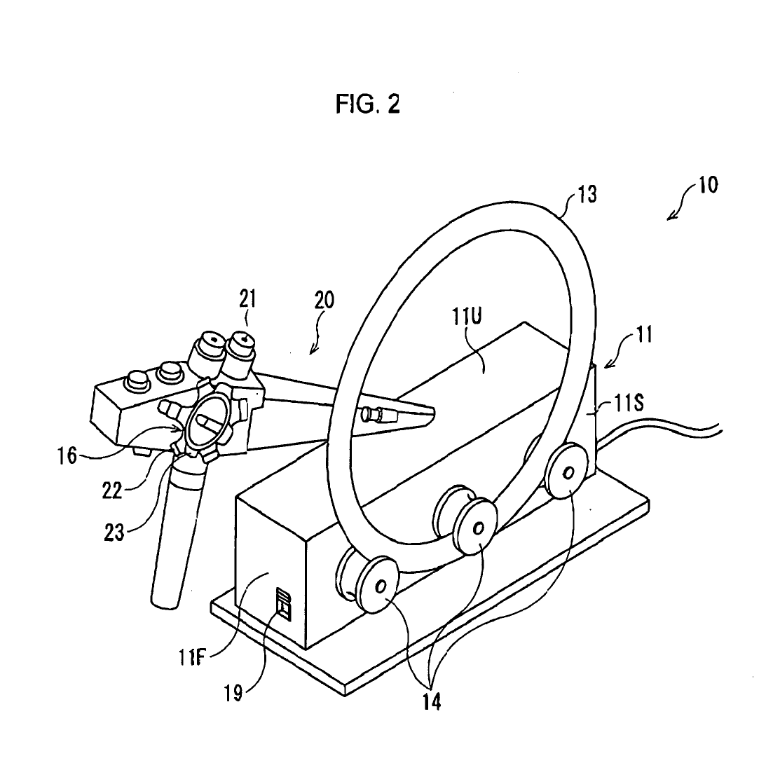

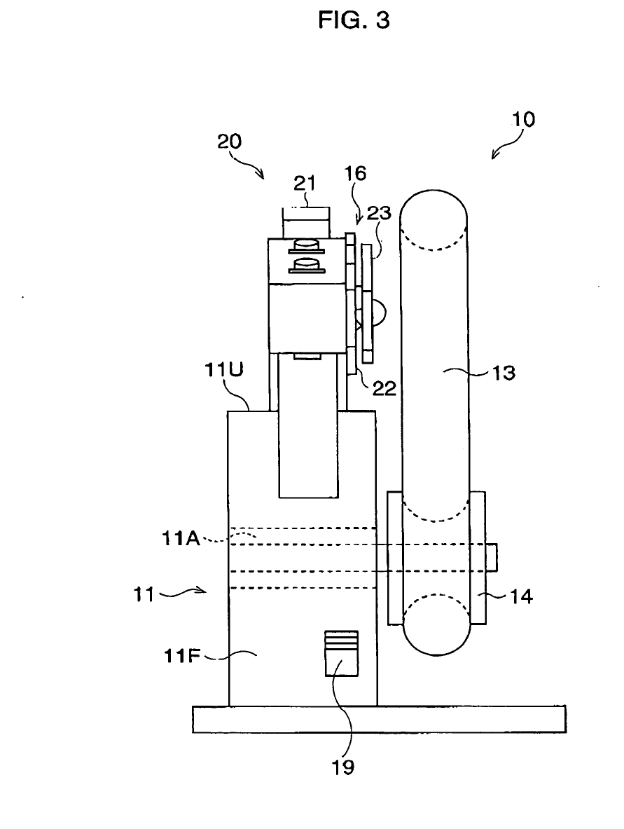

A mode selecting switch 19 is provided on a front surface 11F of the casing 11 (see FIGS. 2 and 3). The mode selecting switch 19 is used for selecting one or a direct generating mode and an indirect generating mode. In the direct generating mode, the moving amount data representing the moving amount of the virtual tip of the endoscope, is generated based on the actual operation amount of the first endless member 13. On the other hand, in the indirect generating mode, the moving amount data is generated based on a proportional operation amount of the first endless member 13, that is proportional to the actual operation amount of the first endless member 13. In this embodiment, the proportional operation amount is set to be one tenth of the actual operation amount. As described above, in the indirect generating mode, the moving amount data is generated based on a proportional operation amount of the first endless member 13, being smaller than the actual operation amount of the first endless member 13, corresponding to the operation amount of a real endoscope. That is, the virtual tip of the endoscope is supposed to be less movable than the real tip of the insertion tube of the endoscope, and the position of the virtual tip can be finely adjusted. Therefore, in the indirect generating mode, a required image can be easily selected from many images existing of a complex area of a subject, by gradually moving the virtual tip of the endoscope, so that the indirect generating mode is especially useful in a case where the affected parts and so on, should be observed.

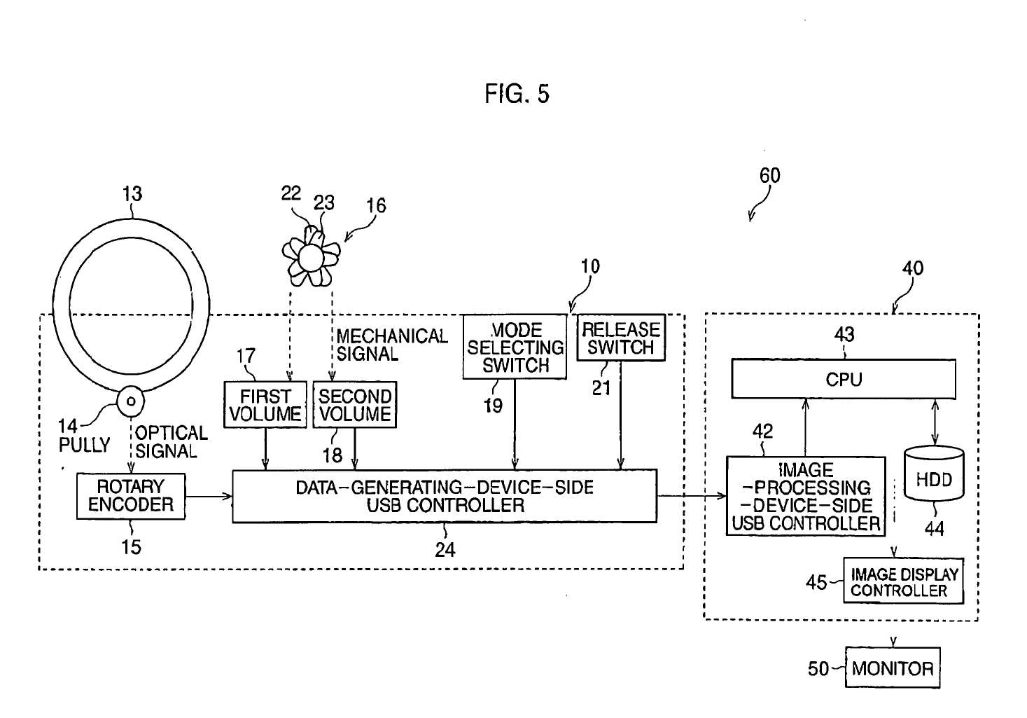

FIG. 5 represents a block diagram of the electronic endoscope 60 including the data generating device 10 and the image processing device 40.

When the pulley 14 rotates due to rotation of the first endless member 13, signals representing a rotation amount of the pulley 14 are transferred to a rotary encoder 15. Therefore, the rotary encoder 15 can detect the rotation amount of the first endless member 13 as an operation amount thereof by the user, and transfer the signals representing the operation amount of the first endless member 13 to a data-generating-device-side USB controller 24. When the first and the second knobs 22, 23 of the angle knob 16 are operated, mechanical signals corresponding to each operation amount are transferred to a first volume 17 and a second volume 18 respectively. These first and second volumes 17, 18 transmit signals representing each of the left and right direction, and the up and down direction of the virtual tip of the endoscope being bent by the simulated operation, according to the received mechanical signals, to the data-generating-device-side USB controller 24.

The data-generating-device-side USB controller 24 generates moving amount data representing the moving amount from a predetermined initial position (reference position) of the virtual tip of the endoscope, by multiplying a predetermined coefficient by the operation amount of the user, represented by the received signals from the rotary encoder 15, that is the amount of rotation of the first endless member 13. Further, the data-generating-device-side USB controller 24 also generates the direction data representing a virtual direction or the virtual tip of the endoscope, based on the received signals from the first and the second volume 17 and 18. The data-generating-device-side USB controller 24 outputs these generated data to an image-processing-device-side USB controller 42 or the image processing device 40.

When the data-generating-device-side USB controller 24 receives signals indicating that the direct generating mode is selected, the moving amount data and the direction data of the virtual tip are generated based on the actual operation amount of the first endless member 13, that is the actual amount of rotation thereof. On the other hand, when the data-generating-device-side USB controller 24 receives signals indicating that the indirect generating mode is selected, the moving amount data and the direction data are generated based on the proportional operation amount calculated by multiplying a weighting coefficient of 0.1 by the actual operation amount of the first endless member 13.

The data-generating-device-side USB controller 24 orders a printer (not shown) connected to the image processing device 40 to print predetermined images based on the signals generated by the depression of the release switch 21, and transfers signals to the image-processing-device-side USB controller 42 for storing the images as 3-D images in a CPU 43 in the image processing device 40.

The image-processing-device-side USB controller 42 transfers the moving amount data and the direction data input by the data-generating-device-side USB controller 24 to the CPU 43. Further, the image-processing-device-side USB controller 42 transfers signals to the CPU 43 for generating position data representing the coordinate system position of the virtual tip after the operation of the first endless member 13, based on the moving amount data. The CPU 43 generates the generating position data representing the position of the virtual tip in a predetermined coordinate system after being moved by an operation of the first endless member 13, by adding the moving amount represented by the moving amount data to the origin in the coordinate system, that is the reference position of the virtual tip before moving.

In a hard disk 44 provided in the image processing device 40, image data of a series of images of subjects such as organs photographed by a capsule, is previously stored. The CPU 43 calculates a range in a subject where the virtual tip can observe, based on the moving amount data and the direction data. Further, the CPU 43 transfers order signals to the hard disk 44 for transferring the image data from the hard disk 44 to the CPU 43, the image data being required to selectively generate a 3-D image of the subject included in the calculated range where the virtual tip can observe, as a partial image.

As a result of this, the image data required for selectively generating the 3-D image of the predetermined part of the subject, is transmitted to the CPU 43 from the hard disk 44, and the 3-D image is generated in the CPU 43 based on the image data. The 3-D image is further transferred from the CPU 43 to a monitor 50 connected to the image processing devise 40, via an image display controller 45 in the image processing device 40. Therefore, the 3-D image of an area in the subject that the virtual tip of the scope can observe after movement caused by the simulated operation by the user, is displayed on the monitor 50.

As described above, in the first embodiment, the data generating device 10 which can generate data for selecting an image of the predetermined part of a subject from a series of images of the subject stored in the image processing device 40, by operations of the first endless member 13 and the scope simulator 20 being similar to those of typical endoscopes, is achieved. Further, the electronic endoscope 60 where an image of a predetermined part of a subject is easily selected and displayed, is achieved by using the generating device 10.

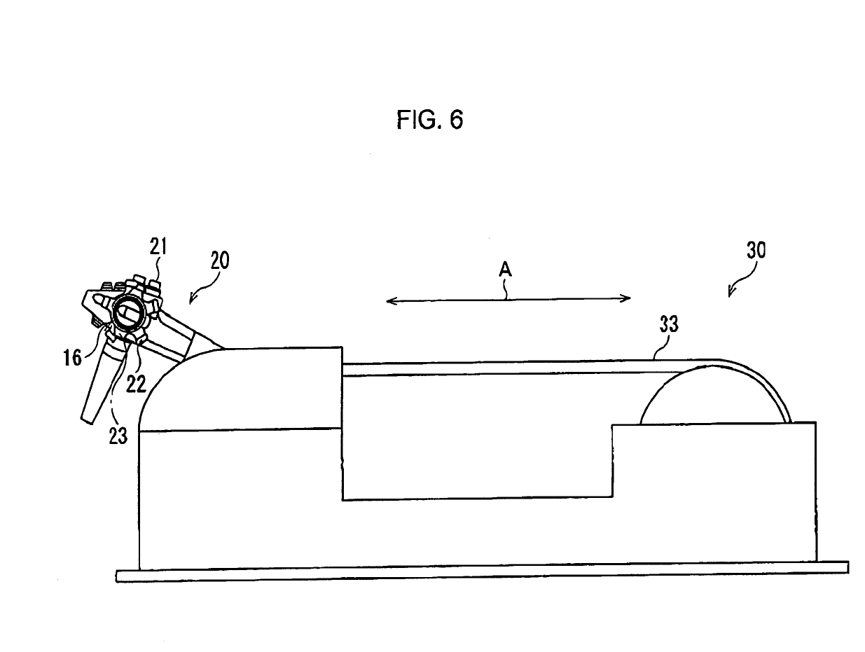

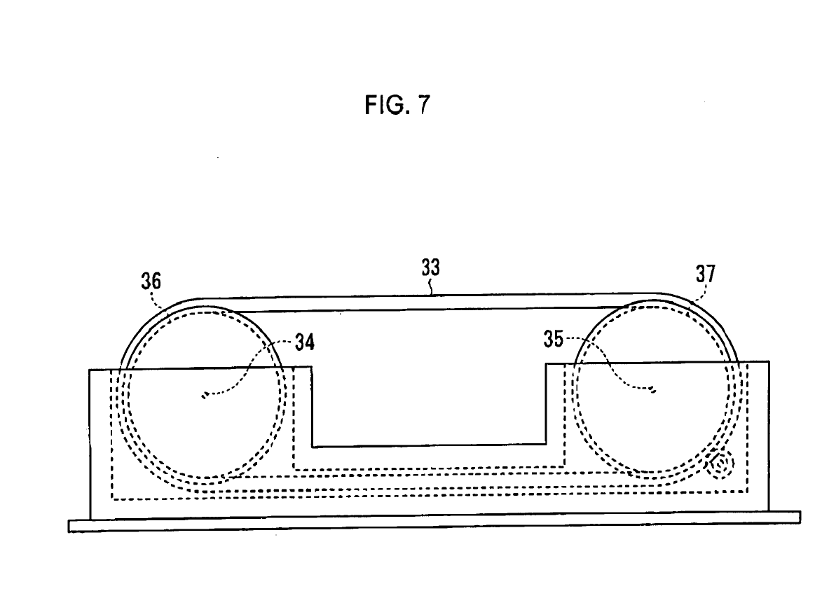

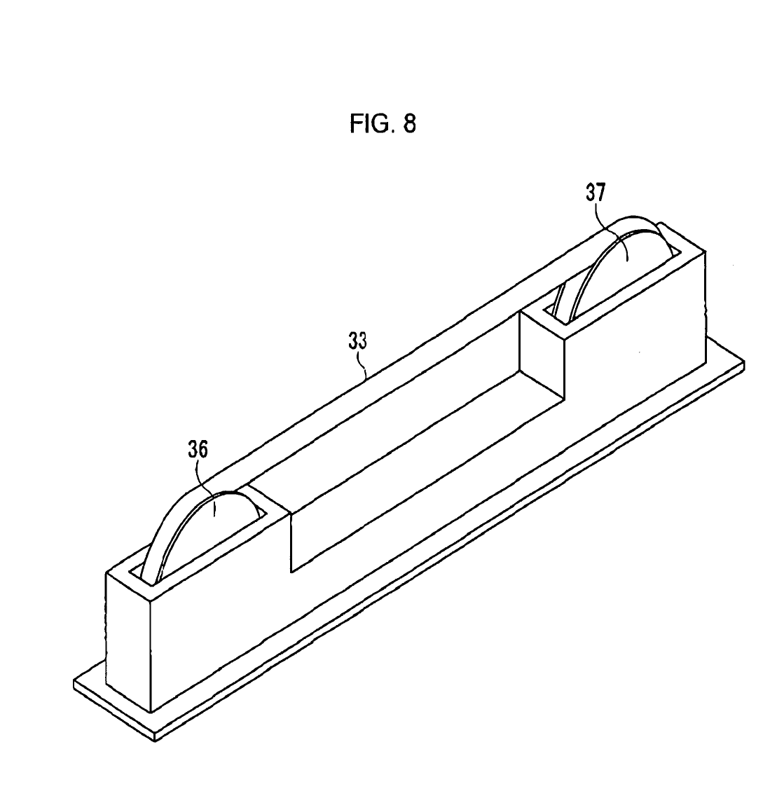

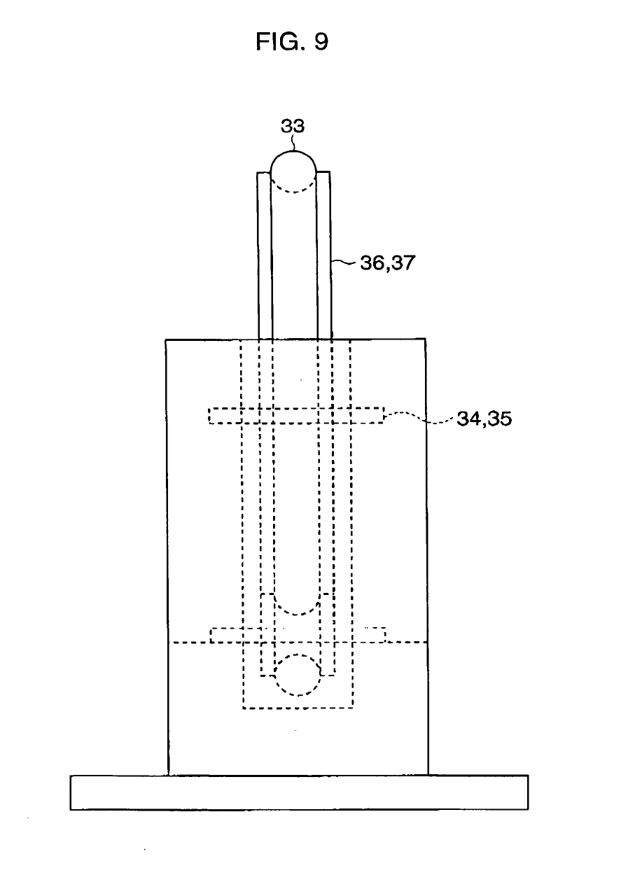

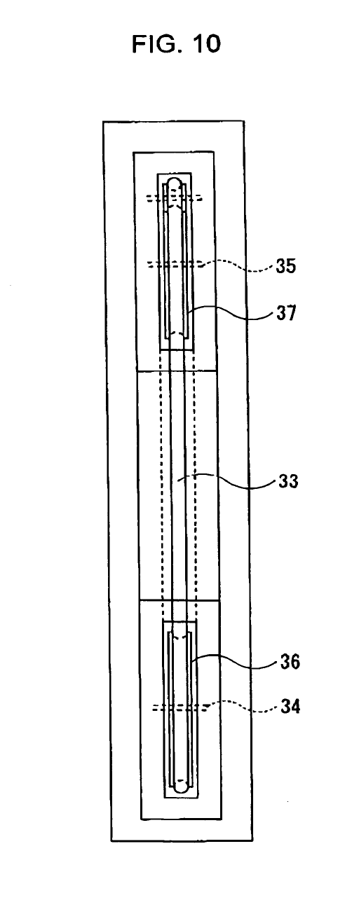

In FIGS. 6 to 10, the data generating device 10 of the second embodiment is shown. In these FIGS. 6 to 10, the same reference numerals are used for the same elements as those in the first embodiment.

In the second embodiment, a second endless member 33 whose end parts have a half-circle shape having approximately the same radius of curvature as the first endless member 33, and whose center part connecting both end parts has a linear shape, is used instead of the first endless member 13. This is the difference between the first and second embodiments. The second endless member 33 is rotationally supported by first and second wheel members 36 and 37 which can respectively rotate around first and second center axes 34 and 35. In the data generating device 30 using the second endless member 33 in this second embodiment, a user operates the second endless member 33 by holding its linear part, so that the second endless member 33 has better operability than the first endless member 13 having round shape only. Further, a user can move the second endless member 33 linearly as arrow A shows, so that the operation of the second endless member 33 is easy especially in the case where the moving amount of the virtual endoscope to be simulated is large. In addition to this, operations for moving the second endless member 33 linearly are close to those when inserting a real scope into a body and pulling it out from a body. Therefore, the data generating device 30 can be easily operated by a user who is familiar with typical endoscopes.

As described above, in the second embodiment, the data generating device 30 generates data for selecting an image of a predetermined part of a subject by easy operations, and outputs the data to the image processing device 40.

The data generating devices 10 and 30 can be applied not only to a capsule endoscope system but other endoscopes such as those that display images generated by an X-ray CT device, an MRI device, and so on, even though they are applied to a capsule endoscope system in both embodiments. In addition to those endoscopes, the data generating devices 10 and 30 can be applied to an endoscope for training in which simulated images of predetermined organs inside body, are stored in an image processing device, instead of the real photographic images of a subject. Further, the images generated based on the position data and direction data, are not limited to a 3-D image.

The CPU 43 can process all the image data for a series of a subject and generate 3-D images of the entire subject first, and then, the 3-D images of a part of the subject to be observed can be displayed based on signals output from the image-processing-device-side USB controller 42, although the CPU 43 processes only the image data of the predetermined part which should be observed by a user based on the signals output from the image-processing-device-side USB controller 42 first, and generates the 3-D images, in both embodiments.

The shapes of the insertion simulators to simulates operations for moving the virtual tip, such as the first and the second endless member 13 and 33, are not limited to those in the embodiments, although the insertion simulators should desirably be endless and rotational to minimize the size of the data generating devices 10 and 30. For example, the insertion simulator an have an annular and not an endless shape by removing a part of the full circle shape, and can be rotational. For the data generating devices, using the scope simulator 20 which enables operations similar to those of typical endoscopes is desirable, however, other scope simulators can be used.

The invention is not limited as described in the preferred embodiments, namely, various improvements and changes may be made to the present invention without departing from the spirit and scope thereof.

The present disclosure relates to subject matter contained in Japanese Patent Application No. 2005-056072 (filed on Mar. 1, 2005) which is expressly incorporated herein, by reference, in its entirety.

1. A data generating device for endoscope observation, comprising;
an annular insertion simulator which simulates insertion of an endoscope into a subject; and
a moving amount data generator that generates moving amount data representing a virtual moving amount of said annular insertion simulator during a simulated insertion, based on an actual operation amount of said annular insertion simulator.

2. The data generating device according to claim 1, wherein said moving amount data represents a virtual moving amount of said annular insertion simulator from a reference position.

3. The data generating device according to claim 1, wherein said annular insertion simulator is an endless loop.

4. The data generating device according to claim 1, wherein a part of said annular insertion simulator is linear.

5. The data generating device according to claim 1, further comprising an operation amount detector that detects said actual operation amount of said annular insertion simulator.

6. The data generating device according to claim 5, wherein said operation amount detector detects amount of rotation of said annular insertion simulator as said actual operation amount, and said moving amount data generator generates said moving amount data by multiplying a predetermined coefficient by said amount of rotation.

7. The data generating device according to claim 1, further comprising a direction data generator that generates direction data representing a virtual direction of a virtual tip of said annular insertion simulator for observing said subject.

8. The data generating device according to claim 7, further comprising a data outputting device that outputs said moving amount data and said direction data to an external device.

9. The data generating device according to claim 7, further comprising a direction adjuster that adjusts said virtual direction.

10. The data generating device according to claim 9, wherein said direction adjuster includes an angle knob to simulate an operation for adjusting a direction of said virtual tip.

11. The data generating device according to claim 9, wherein said annular insertion simulator is provided close to said direction adjuster so that said annular insertion simulator and said direction adjuster can be operated by a user at the same time.

12. The data generating device according to claim 1, further comprising a mode selector that selects one of a first mode where said moving amount data generator generates said moving amount data based on said actual operation amount of said annular insertion simulator, and a second mode where said moving amount data generator generates said moving amount data based on a proportional operation amount of said annular insertion simulator, said proportional operation amount being proportional to said actual operation amount.

13. The data generating device according to claim 12, wherein said proportional operation amount is smaller than said actual operation amount.

14. An endoscope system comprising;
an annular insertion simulator which simulates insertion of an endoscope into a subject;
a moving amount data generator that generates moving amount data representing a virtual moving amount of said annular insertion simulator during a simulated insertion, based on an actual operation amount of said annular insertion simulator;
a position data generator that generates position data representing a position of a virtual tip of said annular insertion simulator for observing said subject, based on a predetermined reference position of said virtual tip and said moving amount data;
an image storage that stores image data of said subject; and
an image generator that generates a partial image of said subject by processing said image data;
wherein said image generator generates said partial image corresponding to a position of said virtual tip, said position being represented by said position data, and said partial image being supposed to observe.

15. The endoscope system according to claim 14, further comprising an image display that displays said partial image.
